(19) United States
(12) Patent Application Publication
(10) Pub. No.: US 2005/0112754 A1
(43) Pub. Date: May 26, 2005

(54) Apparatus and method for amplifying a polynucleotide

(76) Inventors: Dae-Sung Yoon, Gyeonggi-do (KR); You-Seop Lee, Gyeonggi-do (KR); Geun-Bae Lim, Gyeonggi-do (KR); Jae-Chang Lee, Gyeonggi-do (KR); Moon-Hyuck Jung, Gyeonggi-do (KR)

Correspondence Address: CANTOR COLBURN, LLP, 55 GRIFFIN ROAD SOUTH, BLOOMFIELD, CT, 06002, US

(21) Appl. No.: 10/476,036

(22) PCT Filed: Dec. 5, 2002
(86) PCT No.: PCT/KR02/02291

(30) Foreign Application Priority Data
Mar. 9, 2002 (KR) 2002/12730

(51) Int. Cl. C12M 1/00

(52) U.S. Cl. 435/287.2; 435/303.1; 435/288.5; 435/91.2

(57) ABSTRACT

The present invention provides an apparatus for amplifying a polynucleotide, comprising a substrate, a microflow channel system disposed in the substrate and comprising a sample inlet port, a sample flow channel extending from the sample inlet port, and a polynucleotide polymerization reaction chamber in fluid communication with the sample flow channel, a first insulation groove formed around the reaction chamber, and a means for regulating a temperature of the reaction chamber. Accordingly, a multiple chamber device for amplifying a polynucleotide, comprising multiple polymerization reaction chambers formed in a substrate can be manufactured.

### TECHNICAL FIELD

The present invention relates to an apparatus for amplifying a polynucleotide, and more particularly, to an apparatus for amplifying a polynucleotide having multiple chambers in a single substrate and a method for amplifying a polynucleotide.

### BACKGROUND ART

A conventional device for amplifying a polynucleotide comprises at least one reaction tube of 0.2 ml or 0.5 ml, and PCR is conducted by subjecting the tube to an identical temperature cycle. In this case, a target polynucleotide having a different temperature cycle for amplification can not be amplified. Further, there are difficulties in preparing a sample because a sample volume should be at least 0.2 ml.

Most of conventional apparatus for amplifying a polynucleotide include one polymerization reaction tube as disclosed in U.S. Pat. No. 5,955,029 and 6,126,804. Thus, there are difficulties in amplifying a plurality of polynucleotides by using these devices. Moreover, in conventional devices, a polymerization reaction chamber is not thermally insulated from the other parts thereof. Therefore, in a lab-on-a-chip comprising a device for amplifying a polynucleotide, a temperature of each chamber influences a temperature of the other parts thereof. As a result, a temperature of a polymerization chamber has an effect on means for a sample pre-treatment and means for detection. Therefore, in a device for amplifying a polynucleotide having a plurality of reaction chambers and a lab-on-a chip, each chamber should be insulated. Otherwise, it is quite hard to control the temperature of each chamber because of temperature interference.

Daniel et al. introduced an insulation conception to a device for amplifying a polynucleotide (J. H. Daniel et al., Sensor and Actuator, A471, pp. 81-88, 1998). Daniel's device has a mesh structure in which the surrounding of the reaction chamber is etched and has a web-like shape. The device has advantages in aspects of insulation and cooling, but there are difficulties in fabricating various flow channels and electrodes in the device. Therefore, it is difficult to apply the structure to a lab-on-a chip.

### DISCLOSURE OF THE INVENTION

It is an object of the present invention to provide an apparatus for amplifying a polynucleotide having means for thermal insulation.

It is another object of the present invention to provide a multiple chamber apparatus for amplifying a polynucleotide having means for thermal insulation.

It is yet another object of the present invention to provide a method for amplifying a polynucleotide using the apparatus for amplifying a polynucleotide of the present invention.

The present invention provides an apparatus for amplifying a polynucleotide, comprising: a substrate; a microflow channel system disposed in the substrate and comprising a sample inlet port, a sample flow channel extending from the sample inlet port, and a polynucleotide polymerization reaction chamber in fluid communication with the sample flow channel; a first insulation groove formed around the reaction chamber; and a means for regulating a temperature of the reaction chamber.

The present invention provides an apparatus for amplifying a polynucleotide, comprising a substrate and a plurality of unit devices for amplifying a polynucleotide disposed on the substrate, each of the unit device comprising: a microflow channel system disposed in the substrate and comprising a sample inlet port, a sample flow channel extending from the sample inlet port, and a polynucleotide polymerization reaction chamber in fluid communication with the sample flow channel; a first insulation groove formed around the reaction chamber; and a means for regulating a temperature of the reaction chamber.

The present invention also provides a method for amplifying a polynucleotide contained in a sample by conducting PCR, comprising: preparing a biochip comprising a reaction chamber and insulation groove in a substrate; delivering a sample polynucleotide and a reagent for a polymerization reaction; and controlling the temperature of the reaction chamber for PCR.

Further, the present invention provides a method for amplifying a polynucleotide contained in a sample by conducting PCR, comprising: (a) preparing a biochip comprising a substrate and a plurality of unit amplification devices, each unit amplification device comprising a; a microflow channel system disposed in the substrate and comprising a sample inlet port, a sample flow channel extending from the sample inlet port, and a polynucleotide polymerization reaction chamber in fluid communication with the sample flow channel; a first insulation groove formed around the reaction chamber; and a means for regulating a temperature of the reaction chamber, (b) delivering a sample polynucleotide and a reagent for a polymerization reaction to each of the reaction chambers; and (c) independently controlling the temperature of the reaction chambers for PCR.

### BRIEF DESCRIPTION OF THE DRAWINGS

FIG. 1 is a schematic top view illustrating an apparatus for amplifying a polynucleotide according to one embodiment of the present invention;

FIG. 2 is a schematic cross-sectional view illustrating an apparatus for amplifying a polynucleotide according to one embodiment of the present invention;

FIG. 3 is a schematic top view illustrating a multiple chamber apparatus for amplifying a polynucleotide according to another embodiment of the present invention;

FIG. 4 is a graph illustration a temperature increase profile of a reaction chamber according to a groove width;

FIG. 5 is an oscillograph illustrating a potential change of a temperature sensor of a multiple chamber apparatus for amplifying a polynucleotide according to another embodiment of the present invention.

FIG. 6 is an oscillograph illustrating a signal read by a controller corresponding to the potential change according to the temperature sensor in FIG. 5.

FIG. 7 is a graph illustrating a maximum overshoot produced during the temperature regulation process of the apparatus according to another embodiment of the present invention.

FIG. 8 is a graph illustrating errors in a steady state produced during the temperature regulation process of the apparatus according to another embodiment of the present invention.

FIG. 9 is a photograph showing a result of gel electrophoresis for a PCR product amplified by using the multiple chamber apparatus according another embodiment of the present invention.

### BEST MODE FOR CARRYING OUT THE INVENTION

The apparatus of the present invention will be described in further detail with reference to the accompanying drawings.

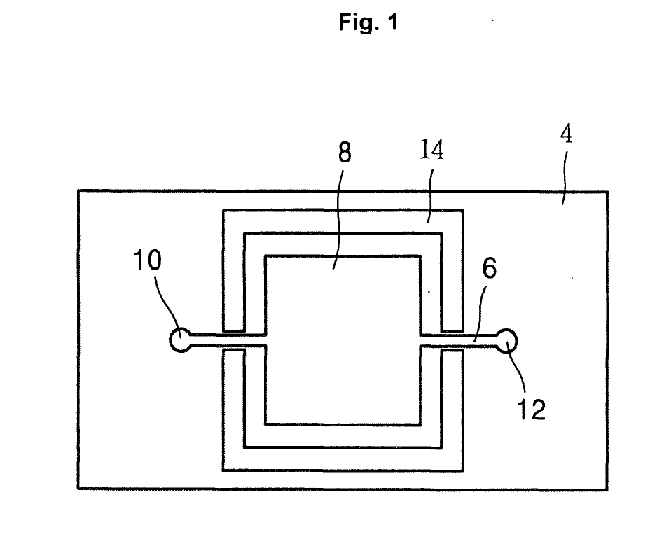

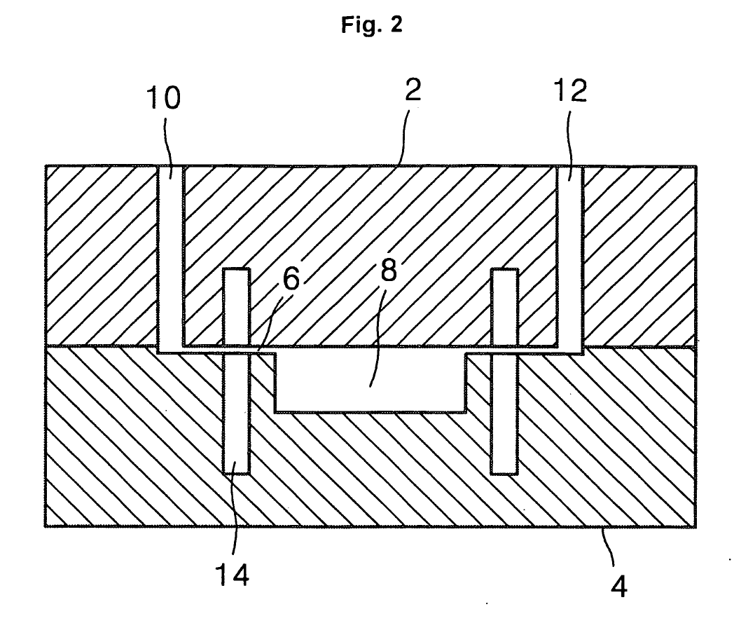

FIGS. 1 and 2 are a schematic top view and cross-sectional view illustrating an apparatus for amplifying a polynucleotide according to one embodiment of the present invention.

According to FIG. 1, the apparatus includes a substrate 4; a microflow channel system; a first groove 14; and temperature controller regulating the temperature of the chamber (not shown). The microflow channel system and the first groove 14 are fabricated in the substrate 4. The microflow channel system is consisting of a sample inlet port 10, a sample flow channel 6 and a polymerization reaction chamber 8. The first groove 14 is microfabricated around the polymerization reaction chamber 8. The temperature controller is disposed on the lower surface of the substrate 4.

According to FIG. 2, the substrate is consisting of upper substrate 2 and lower substrate 4. The inlet port 10, the first groove 14 and the outlet port 12 are fabricated in the upper substrate 2. The sample flow channel 6, the first groove 14 and the polymerization reaction chamber 8 are fabricated in the lower substrate 4. The apparatus is made by attaching the upper substrate 2 and the lower substrate 4.

A sample containing a target polynucleotide is injected into the inlet port 10 and delivered into the polymerization reaction chamber 8 through a sample flow channel 6. The PCR is conducted within the polymerization reaction chamber 8. The PCR temperature cycle is controlled by the temperature controller. The PCR product obtained by the reaction is released into outlet port 12 through the sample flow channel 6.

Examples of materials for the substrate include silicon, glass, polycarbonate, polydimethylsiloxane and polymethylmethacrylate. The microflow channel system has the width, the depth and the height of about 0.1 μm to 500 μm, respectively. Preferably, the polymerization reaction chamber has the width, the depth and the height of about 2.0 μm to 500 μm, and more preferably about 3.0 μm to 500 μm, respectively. But, the size of the chamber is not limited to these specific ranges, and a rather big chamber having the width, the depth and the height of about 1 to 500 mm respectively can be used. The reaction chamber can have any kind of shape including a cube, a rectangular parallelepiped, a cylinder shape.

The first groove may have the width of about 0.3 mm to 3 mm. And the first groove may have the depth of about 200 μm to 290 μm in case that a silicon substrate has a depth of 300 μm and about 200 μm to 490 μm in case that a silicon substrate has a depth of 500 μm. But, the size of the first groove is not limited to these specific ranges.

The temperature controller for regulating the temperature of the chamber may comprise a heater and a temperature sensor for thermally regulating the PCR temperature cycle required for a hybridization and dehybridization. The temperature of the chamber can be controlled by supplying one or more electrical heaters around the chamber, or by applying a pulse laser or other electromagnetic energies to the chamber. In addition, the apparatus for amplifying a polynucleotide may comprise a cooling member which can be any structure conventionally used for the purpose of cooling. An electrode for the heaters may be disposed under the chamber or around the chamber. Preferably, the electrode is disposed on a lower surface of the substrate having the chamber.

The apparatus may further comprise a detector for detecting an amplified polynucleotide and an outlet port 12 for releasing the amplified poynucleotide. The detector may use a conventional means for detecting a polynucleotide, for example, means for measuring the resistance of a fluid flow, and fluorescent or spectrophometric detection means. The outlet port can be formed as a part of a microflow channel system of the present invention, and can be in fluid communication with the chamber.

The apparatus for amplifying a polynucleotide may further comprise a cell lysis means. The cell lysis means can be in fluid communication with the reaction chamber for casusing a lysis of the cell used as a sample.

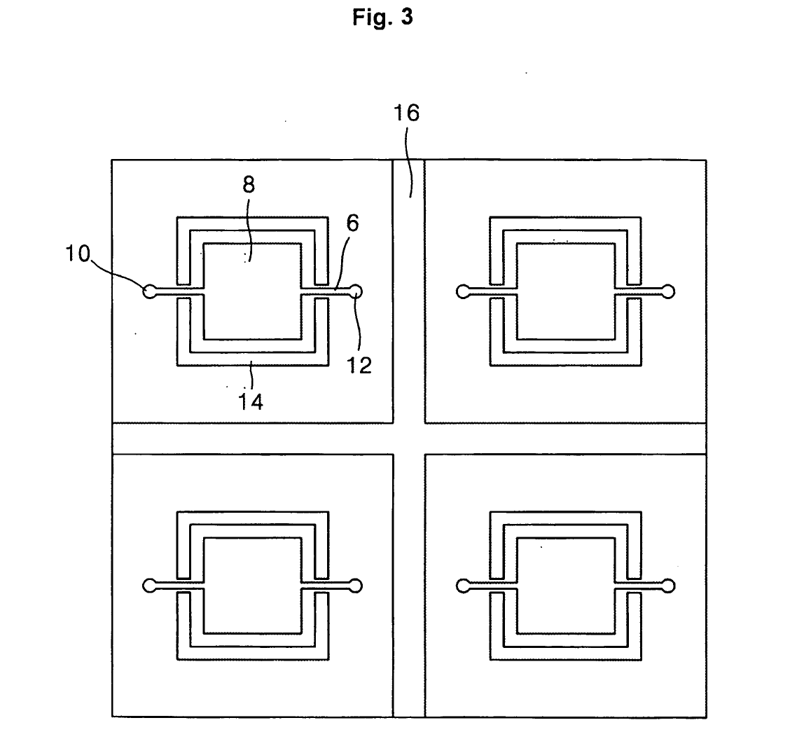

FIG. 3 is a schematic top view illustrating a multiple chamber apparatus for amplifying a polynucleotide according to another embodiment of the present invention.

As shown in FIG. 3, the multiple chamber apparatus for amplifying a polynucleotide includes four Unit devices for amplifying a polynucleotide. Four unit devices are microfabricated in a single substrate. Each unit device for amplifying a polynucleotide comprises a substrate 4; a microflow channel system; a first groove 14; and a temperature controller(not shown). The microflow channel system is consisting of a sample inlet port 10, a sample flow channel 6 and a polymerization reaction chamber 8. The first groove 14 is fabricated around the polymerization reaction chamber 8. The temperature controller can be disposed on the lower surface of the substrate 4. Alternatively, the temperature controller can be disposed under the polymerization reaction chamber 8 in the substrate 4. Since the multiple-chamber apparatus is fabricated in a single substrate, a plurality of polynucleotides contained in a sample can be simultaneously amplified in separate chambers temperature of which are independently controlled.

The apparatus of one embodiment of the invention may further comprise a second groove 16 defining a boundary of each unit device for amplifying a polynucleotide. The reaction chamber of each unit device can be independently thermally regulated and thus each unit device can independently conduct a PCR by the first insulation groove 14 fabricated around the chamber and the second insulation groove 16 fabricated between unit devices.

The multiple chamber apparatus for amplifying a polynucleotide may comprise means for controlling the temperature of the reaction chambers such that PCR in respective reaction chambers are conducted according to a same time schedule or a different time schedule. The means for thermally controlling the reaction chamber may comprise a controller, a power supplier, a temperature sensor, and a heater. The controller generates a control signal based on a control information on a preselected control temperature and control time, and information on a real temperature supplied from the temperature sensor and provides the control signal to the power supplier. The power supplier provides a power to the heater according to the control signal. The heater receives a power from the power supplier to produce heat, the temperature sensor measures a real temperature in the reaction chamber and supplies the information on the real temperature to the controller. The supply of the control signal from the controller to the power supplier can be made by using a PID method or on/off computation method. If the on/off computation method is used, a MOSFET can be used.

The apparatus for amplifying a polynucleotide can be manufactured by a variety of method, particularly using a photolithography process which is generally used in semiconductor manufacturing industries.

A photolithography process for manufacturing the apparatus for amplifying a polynucleotide according to one embodiment of the invention is described in detail. A first substrate such as silicon is coated with an oxide film on its surface, and then a sample flow channel, a polymerization reaction chamber, and an insulation groove are patterned by using a photomask. The surface are etched to a desired depth by using an oxide film pattern and a wet etching or a dry etching including a reactive ion etching. If necessary, these patterning process and etching process can be repeated several times. A lower surface of the first substrate is subjected to patterning and etching, and coated with a metallic film such as platinum, gold, nickel, and copper to form an electrode. A second substrate such as silicon is coated with an oxide film on its surface, and then a sample inlet port, an insulation groove, and an outlet port are patterned by using a photomask, and then etched to a desired depth. The first and second substrates are attached to complete an apparatus for amplifying a polynucleotide of one embodiment of the present invention. The attachment can be made by using a process including cathode sealing, fluoride sealing, heat sealing or polymer sealing.

One or more heaters and sensors are placed on the apparatus for amplifying a polynucleotide of one embodiment of the present invention. The sensor maintains a temperature of the chamber at a constant level, measures a potential induced from the temperature and determines a relationship between the temperature and the potential. The controller converts a particular potential measured by the sensor into a particular temperature by using the relationship and displays the particular temperature.

The present invention is further described by the following examples, but it should not be confined or limited to these examples.

### EXAMPLE 1

### Temperature Increase Profile and Temperature Distribution of a Chamber According to Presence or Absence of an Insulation Groove in an Apparatus for Amplifying a Polynucleotide

(1) Measurement of Temperature Distribution

A temperature distribution was measured for an apparatus for amplifying a polynucleotide having an insulation groove around the chamber as shown in FIG. 1 while heating the reaction chamber upto 410K. As a control, an apparatus for amplifying a polynucleotide without an insulation groove was used. The apparatus for amplifying a polynucleotide without an insulation groove is the same as the apparatus described in FIG. 1 except that it has no insulation groove. The groove has a width of 1 mm and a depth of 250 μm.

Power consumption for raising the temperature upto 410K was about 2.8 W in an apparatus for amplifying a polynucleotide having an insulation groove, while 4W in a control apparatus. Therefore, the power consumption was reduced by 30%, and thus an insulation effect was achieved by fabricating an insulation groove.

(2) Measurement of Temperature Increase Profile

A temperature increase profile was measured for an apparatus for amplifying a polynucleotide having an insulation groove around the chamber as shown in FIG. 1 while supplying a constant power, 4W to the reaction chamber. Three apparatuses for amplifying a polynucleotide having insulation groove were used with the same groove depth of 250 μm, and a different groove width of 100 μm, 1,000 μm and 4,000 μm, respectively. As a control, an apparatus for amplifying a polynucleotide without an insulation groove was used. The apparatus for amplifying a polynucleotide without an insulation groove is the same as the apparatus described in FIG. 1 except that it has no insulation groove.

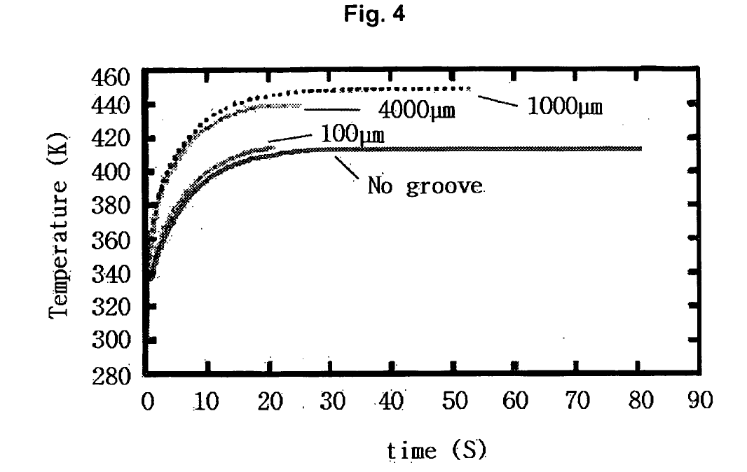

The result is shown in FIG. 4. As shown in FIG. 4, the temperature is increased more rapidly and the final equilibrium temperature is higher in the apparatus having a groove than in the control apparatus. In addition, the rate of temperature increase is proportional to the width of the groove. But, when the width is greater than about 1 mm, there is no further change in the rate of temperature increase and equilibrium temperature.

### EXAMPLE 2

### Temperature Regulation in an Apparatus for Amplifying a Polynucleotide having Multiple Reaction Chambers

In this example, an apparatus for amplifying a polynucleotide having four chambers and a temperature sensor of a platinum thin film as shown in FIG. 3 was used, and the temperature of the reaction chamber was controlled.

3.6 μl of a PCR reaction solution was added into the sample inlet port 10 and the sample flow channel 6, and then to the polymerization reaction chamber 8 (FIG. 3). Temperature control information for the temperature cycles of 30 secs at 55° C., 30 secs at 72° C., 30 secs at 90° C., and 30 secs at 95° C. was input into the controller and the power controller was driven.

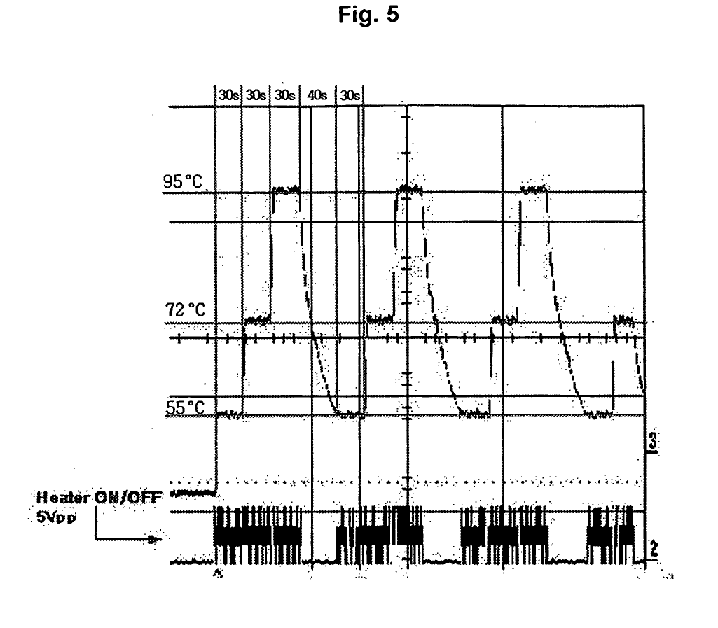

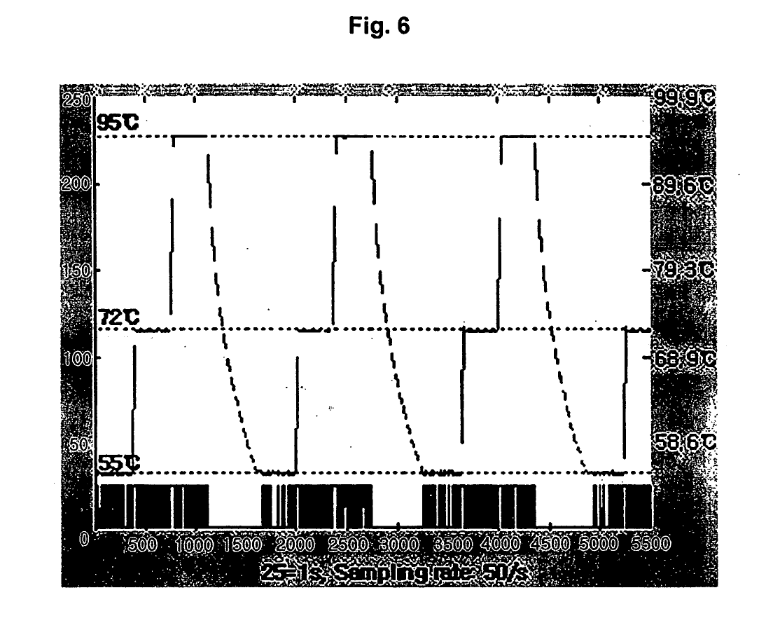

FIG. 5 is an oscillograph illustrating a potential change of a temperature sensor of a multiple chamber apparatus for amplifying a polynucleotide according to one embodiment of the present invention. In FIG. 5, x axis represents the time and y axis represents the potential. Real temperature and maintenance time corresponding to the respective potential were also represented. FIG. 6 is an oscillograph illustrating a signal, read by a controller, corresponding to the potential change of the temperature sensor. The bottom part in the FIGS. 5 and 6 represent an on/off operation.

As shown in FIGS. 5 and 6, a computer, corresponding to a controller, consistently recognized the output potential of a platinum film temperature sensor. These results indicate that the temperature of the apparatus for amplifying a polynucleotide having multiple reaction chambers can be consistently regulated.

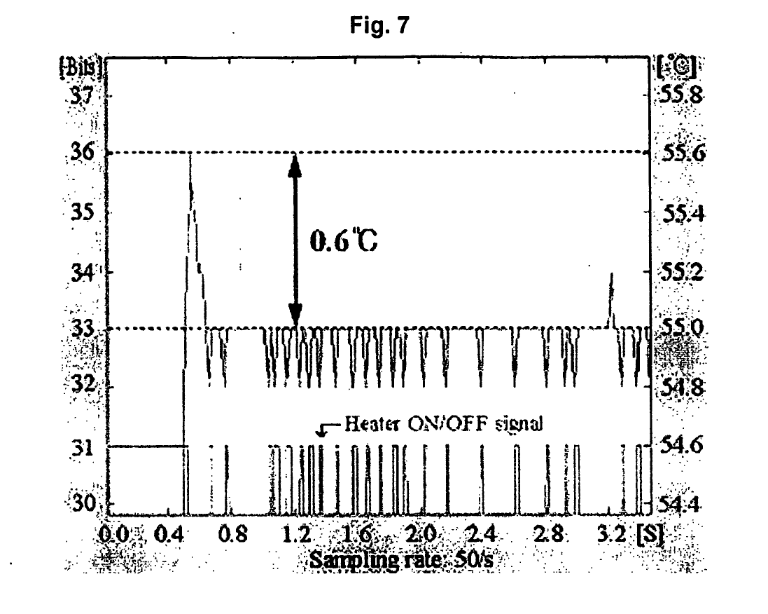

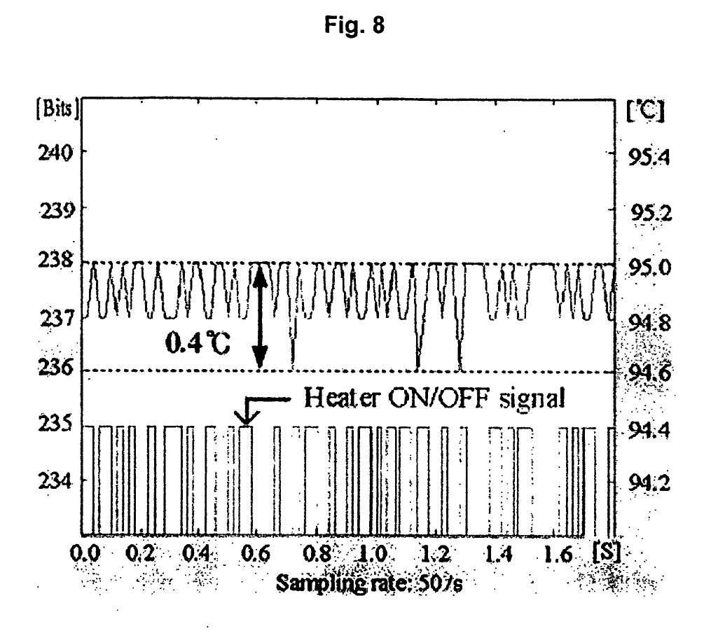

FIGS. 7 and 8 illustrate an overshoot of the heater and the steady state error when the reaction chamber of the apparatus of one embodiment of the invention was heated from room temperature to 55° C. and maintained at that temperature. As shown in FIGS. 7 and 8, the overshoot is less than about 0.6° C., and the steady state error is about ±0.4° C. The rate of temperature increase is 6.7° C./sec. The apparatus for amplifying a polynucleotide of one embodiment of the present invention has improved heating and cooling characteristics and similar steady state error value in comparison with the conventional bulk PCR machine using a 0.2 ml reaction tube.

### EXAMPLE 3

### PCR using an Apparatus for Amplifying a Polynucleotide having a Multiple Reaction Chamber

A PCR was conducted by using an apparatus for amplifying a polynucleotide having four chambers as shown in FIG. 3 and a platinum film temperature sensor.

The PCR using said apparatus was performed by using a PCR Core system 11 (Promega Co., Madison, U.S.A). A premix containing upstream and downstream control primers, dNTP, salts, DNA polmerase, and the plasmid DNA sample was prepared. The premix was supplied into the sample inlet port and delivered to the polymerization reaction chamber of 2.6 μl volume through the sample flow channel. The sample inlet port and outlet port were sealed by using an epoxy material. The PCR temperature cycle included 30 secs at 55° C., 30 secs at 72° C., and 30 secs at 95° C., and 30 cycles were repeated for PCR.

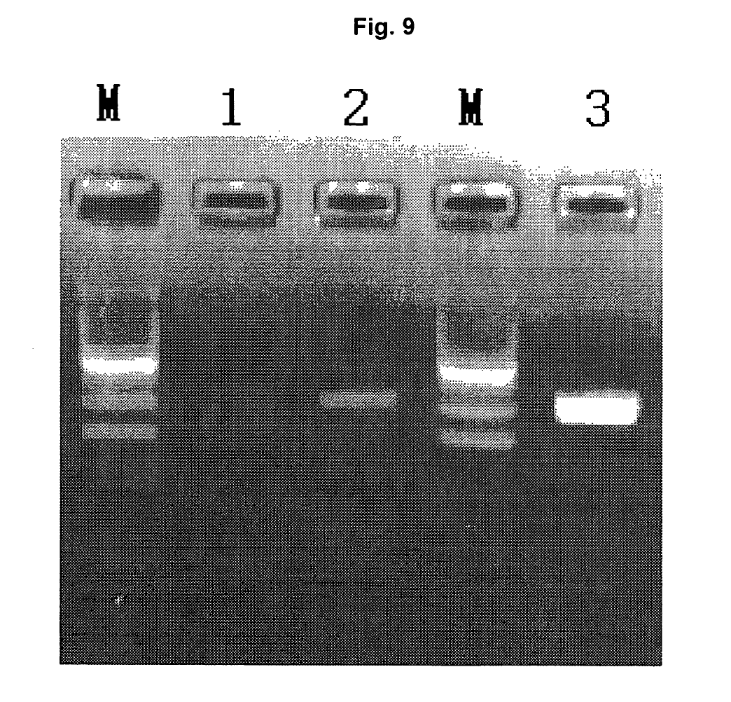

FIG. 9 is a photograph showing a result of gel electrophoresis for a PCR product amplified by using the multiple chamber apparatus of one embodiment of the present invention. In FIG. 9, lane 1 represents a negative control, lane 2 represents an amplified product obtained by using the apparatus having multiple reaction chamber of one embodiment of the invention, lane 3 represents an amplified product obtained by using a control apparatus without a groove, and M represents a size marker. As shown in FIG. 9, the result of amplification product obtained by using the apparatus having a multiple reaction chamber of one embodiment of the invention was similar to that of amplification obtained by using a control apparatus without a groove for amplifying a polynuleotide.

Industrial Applicability

According to the apparatus for amplifying a polynucleotide of the invention, the temperature controllability of the reaction chamber can be increased and power consumption can be reduced by forming an insulation groove on a substrate.

According to the apparatus of the invention, an apparatus for amplifying a polynucleotide having a plurality of reaction chambers in a single substrate can be made by forming an insulation groove on the substrate.

According to the apparatus for amplifying a polynucleotide having a plurality of reaction chambers of one embodiment of the invention, the temperature of the reaction chambers can be independently regulated.

According to the method for amplifying a polynucleotide, a large amount of genes can be amplified with high speed and low cost.

1. An apparatus for amplifying a polynucleotide, comprising:
a substrate;
a microflow channel system disposed in the substrate and comprising a sample inlet port, a sample flow channel extending from the sample inlet port, and a polynucleotide polymerization reaction chamber in fluid communication with the sample flow channel;
a first insulation groove formed around the reaction chamber; and
a means for regulating a temperature of the reaction chamber.

2. The apparatus of claim 1, wherein the depth of the sample flow channel and the polymerization reaction chamber are about 0.1 to 500 μm.

3. The apparatus of claim 1, wherein the width of the sample flow channel and the polymerization reaction chamber are about 0.1 to 500 μm.

4. The apparatus of claim 1, further comprising a cell lysis means for lysing a cell sample, the cell lysis means being in fluid communication with the reaction chamber.

5. The apparatus of claim 1, wherein the first groove has a width of about 0.3 mm to 3 mm.

6. The apparatus of claim 1, wherein the first groove has a depth of about 200 μm to 290 μm in case that a silicon substrate has a depth of 300 μm or a depth of about 200 μm to 490 μm in case that a silicon substrate has a depth of 500 μm.

7. An apparatus for amplifying a polynucleotide, comprising a substrate and a plurality of unit devices for amplifying a polynucleotide disposed on the substrate, each of the unit device comprising;
a microflow channel system disposed in the substrate and comprising a sample inlet port, a sample flow channel extending from the sample inlet port, and a polynucleotide polymerization reaction chamber in fluid communication with the sample flow channel;
a first insulation groove formed around the reaction chamber; and a means for regulating a temperature of the reaction chamber.

8. The apparatus of claim 7, wherein the depth of the sample flow channel and the polymerization reaction chamber are about 0.1 to 500 μm.

9. The apparatus of claim 7, wherein the width of the sample flow channel and the polymerization reaction chamber are about 0.1 to 500 μm.

10. The apparatus of claim 7, wherein the each unit device is further comprising a cell lysis means for lysing a cell sample, the cell lysis means being in fluid communication with the reaction chamber

11. The apparatus of claim 7, wherein the first groove has a width of about 0.3 mm to 3 mm.

12. The apparatus of claim 7, wherein the first groove has a depth of about 200 μm to 290 μm in case that a silicon substrate has a depth of 300 μm or a depth of about 200 μm to 490 μm in case that a silicon substrate has a depth of 500 μm.

13. The apparatus of claim 7, further comprising a second insulation groove for defining a boundary of each unit device for amplifying a polynucleotide.

14. A method for amplifying a polynucleotide contained in a sample by conducting PCR, comprising:
preparing a biochip comprising a reaction chamber and insulation groove in a substrate;
delivering a sample polynucleotide and a reagent for a polymerization reaction; and
controlling the temperature of the reaction chamber for PCR.

15. A method for amplifying a polynucleotide contained in a sample by conducting PCR, comprising:
preparing a biochip comprising a substrate and a plurality of unit amplification devices, each unit amplification device comprising;
a microflow channel system disposed in the substrate and comprising a sample inlet port, a sample flow channel extending from the sample inlet port, and a polynucleotide polymerization reaction chamber in fluid communication with the sample flow channel;
a first insulation groove formed around the reaction chamber; and
a means for regulating a temperature of the reaction chamber,
delivering a sample polynucleotide and a reagent for a polymerization reaction to each of the reaction chambers; and
independently controlling the temperature of the reaction chambers for PCR.

16. The method of claim 15, wherein the biochip further comprising a second insulation groove defining a boundary of each unit amplification device.

17. The method of claim 15, wherein independently controlling includes independently controlling the temperature of the reaction chambers at the same time schedule.

18. The method of claim 15, wherein independently controlling includes independently controlling the temperature of the reaction chambers at a different time schedule.
